(19) United States
(12) Patent Application Publication
(10) Pub. No.: US 2025/0283798 A1
(43) Pub. Date: Sep. 11, 2025

(54) Measuring Rock Permeability

(71) Applicant: Saudi Arabian Oil Company, Dhahran (SA)

(72) Inventors: Jilin Jay Zhang, Houston, TX (US); Hui-Hai Liu, Houston, TX (US)

(21) Appl. No.: 18/598,383

(22) Filed: Mar. 7, 2024

(51) Int. Cl. G01N 15/08; G01N 33/24

(52) U.S. Cl. CPC G01N 15/0826; G01N 15/0806; G01N 33/24

(57) ABSTRACT

Systems and methods for measuring rock permeability include positioning a core sample in a core sample assembly that is enclosed in a pressurized container with a flow inlet, a flow outlet, and a pressurized fluid inlet fluidly coupled to a pressurized fluid reservoir that includes a pressurized fluid pump; performing a low pore pressure test operation on the core sample; sequentially performing at least three high pore pressure test operations on the core sample measuring an inlet pressure at the flow inlet, measuring an outlet pressure at the flow outlet, and measuring a confining pressure within the pressurized container; and determining a permeability of the core sample based at least in part on at least one of the measured inlet pressures, at least one of the measured outlet pressures, and at least one of the measured confining pressures.

### TECHNICAL FIELD

The present disclosure relates to measuring rock permeability.

### BACKGROUND

Pore pressure of a subsurface reservoir, such as an unconventional reservoir, can decrease with hydrocarbon production. This decrease in pore pressure can result in an increase in effective stress, or the difference between the overburden pressure and the pore pressure. In some cases, there is a linear relationship between the logarithm of the permeability and the effective stress that is important for predicting the hydrocarbon production.

### SUMMARY

When the pore pressure in a subsurface reservoir decreases to a certain level, the molecules in the gas flow become subject to diffusion as the collisions among the gas molecules become negligible (e.g., compared to the collisions between gas molecules and the pore wall) when the size of the rock pores (e.g., pore throats) are close to the same size as the length of the mean free path of the gas molecules. For example, in shale samples, the pore throat sizes can be on the order of nanometers or tens of nanometers which is close to the mean free path of gas molecules at low gas pressures (e.g., tens of nanometers). The measured permeability (or apparent permeability) at low gas pressure can be larger than that of a liquid under the same stress conditions. At low gas pressure, the measured permeability can be heavily affected by two competing processes, the compaction process (e.g., increasing effective stress) and the diffusion process. These two processes affect the measured permeability in opposing directions. The effects of both processes on permeability can be measured in a laboratory setting.

This disclosure describes systems and methods for measuring rock permeability. A core sample is positioned in a core sample assembly that is enclosed in a pressurized container, the pressurized container includes a flow inlet, a flow outlet, and a pressurized fluid inlet fluidly coupled to a pressurized fluid reservoir that includes a pressurized fluid pump. A low pore pressure test operation is performed on the core sample. The low pore pressure test operation includes flowing a test fluid into the flow inlet, flowing the test fluid out of the flow outlet, and flowing a pressurized fluid into the pressurized container from the pressurized fluid reservoir. At least three high pore pressure test operations are sequentially performed on the core sample, each of the at least three high pore pressure test operations includes flowing the test fluid into the flow inlet, flowing the test fluid out of the flow outlet, and flowing a pressurized fluid into the pressurized container from the pressurized fluid reservoir. For the low pore pressure test operation and each of the at least three high pore pressure test operations, an inlet pressure at the flow inlet, an outlet pressure at the flow outlet, and a confining pressure within the pressurized container are measured. A permeability of the core sample based at least in part on at least one of the measured inlet pressures, at least one of the measured outlet pressures, and at least one of the measured confining pressures is determined.

Implementations of the systems and methods of this disclosure can provide various technical benefits. Using large pressure drops across the core sample reduces the time of each test operation in comparison with traditional steady-state methods. Using a low pore pressure test operation can measure the effects of diffusion on the permeability of the core sample. Diffusion can be an important contribution to the permeability for low pore pressures and small pore throat sizes. Using large pressure differentials reduces the time to complete the tests as compared with using smaller pressure differentials. Fluid and rock properties along the flow path within the core sample represent actual conditions as compared with using uniform properties for the entire flow path. Using high pore pressure tests and a low pore pressure test addresses the effects of both compaction and diffusion to provide a complete solution for the core sample with large ranges of confining pressure and pore pressure. The permeability along the length of the flow path is determined based on the size of the pore throat along the flow path in the core sample.

The details of one or more implementations of the subject matter described in this disclosure are set forth in the accompanying drawings and the description below. Other features, aspects, and advantages of the subject matter will become apparent from the description, the drawings, and the claims.

### DESCRIPTION OF THE DRAWINGS

FIG. 1 is a schematic diagram of a core sample test system.

FIG. 2 is a flowchart of an example method performed with or by the core sample test system of FIG. 1.

FIGS. 3A-3B are tables that describes measurements taken during a method for testing a core sample.

FIG. 4 is a table comparing permeability values with and without diffusion for several pore pressures.

FIG. 5 illustrates hydrocarbon production operations that include field operations and computational operations.

FIG. 6 is a block diagram illustrating an example computer system used to provide computational functionalities associated with described algorithms, methods, functions, processes, flows, and procedures according to some implementations of the present disclosure.

Like reference symbols in the various drawings indicate like elements.

### DETAILED DESCRIPTION

This specification describes systems and methods for measuring rock permeability. A core sample is positioned in a core sample assembly that is enclosed in a pressurized container, the pressurized container includes a flow inlet, a flow outlet, and a pressurized fluid inlet fluidly coupled to a pressurized fluid reservoir that includes a pressurized fluid pump. A low pore pressure test operation is performed on the core sample. The low pore pressure test operation includes flowing a test fluid into the flow inlet, flowing the test fluid out of the flow outlet, and flowing a pressurized fluid into the pressurized container from the pressurized fluid reservoir. At least three high pore pressure test operations are sequentially performed on the core sample, each of the at least three high pore pressure test operations includes flowing the test fluid into the flow inlet and flowing the test fluid out of the flow outlet, and flowing a pressurized fluid into the pressurized container from the pressurized fluid reservoir. For the low pore pressure test operation and each of the at least three high pore pressure test operations, an inlet pressure at the flow inlet, an outlet pressure at the flow outlet, and a confining pressure within the pressurized container are measured. For example, the low pore pressure test operation and each of the at least three high pore pressure test operations are conducted as steady state tests with a large pressure differential, e.g., a large difference between the inlet and outlet pressures. A permeability of the core sample based at least in part on at least one of the measured inlet pressures, at least one of the measured outlet pressures, and at least one of the measured confining pressures is determined.

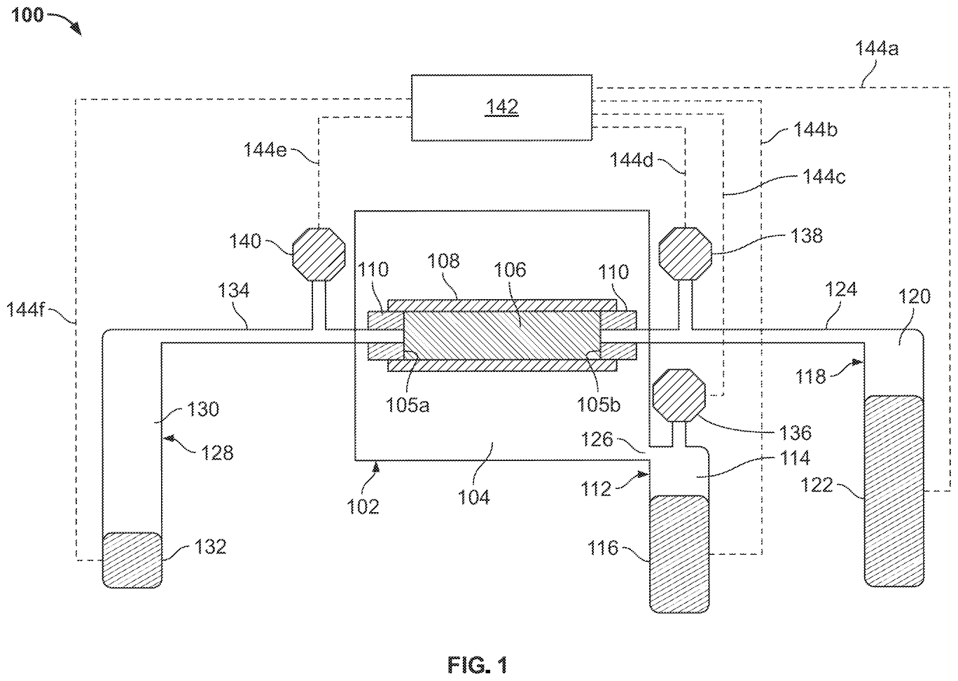

FIG. 1 is a schematic diagram of an example core sample test system 100. In some aspects, the core sample test system 100 can be operated to determine one or more characteristics or properties of a core sample 106 that is positioned within the system 100. Such characteristics include, for example, permeability, pressure dependent characteristics, characteristic pore throat equivalent parameters, as well as poroelastic characteristics. In some aspects, the core sample 106 includes a shale rock sample taken from a subsurface formation; alternatively other types of rocks may also be used as the core sample 106, including rock samples from conventional and unconventional reservoirs.

As shown in FIG. 1, core sample test system 100 includes a core sample assembly 110 in which the core sample 106 is placed and secured. The core sample assembly 110 (or sample stack) includes a sleeve 108 that encircles the core sample 106, which in some aspects, is a cylindrical core sample with a diameter of 1 to 1.5 inches. In some aspects, prior to placing the core sample 106 within the core sample assembly 110, the core sample 106 is cut to between 1 and 2 inches long and pre-processed, e.g., to remove mobile water and hydrocarbon fluids therefrom. In some examples, the pre-processing includes trimming and polishing the end faces of the core sample 106 such that the two end faces are parallel to each other and perpendicular to the axis of the cylindrical core sample 106.

The core sample assembly 110 (including the core sample 106) is placed in a pressurized container 102 that defines a volume 104 in which the holder 110 is placed. As shown in this example, a pressurized fluid reservoir 112 (also called a confining reservoir) is fluidly coupled to the pressurized container 102 through a pressurized fluid inlet 126. Pressurized fluid reservoir 112 also includes or is in fluid communication with a pressurized fluid pump 116 that is operable to circulate a pressurized fluid 114 (e.g., a gas or other fluid) through the pressurized fluid inlet 126 and into the volume 104 to, e.g., controllably change or maintain a pressure of the volume 104 (sometimes called a confining pressure). Note the confining pressurized fluid is physically separated from the pore fluid that flows through the core sample.

A flow inlet 134 is fluidly coupled to the core sample assembly 110 (and thus the core sample 106) through the pressurized container 102 with the two fluids (e.g., confining fluid and pore fluid) physically separated. The flow inlet 134 is also fluidly coupled to an upstream fluid reservoir 128. Upstream fluid reservoir 128 also includes or is in fluid communication with an upstream fluid pump 132 that is operable to circulate a test fluid 130 (e.g., a gas or other fluid, which may be called pore fluid) through the flow inlet 134 and into the core sample assembly 110 to, e.g., controllably change or maintain a pressure at a first end (e.g., an upstream end) of the core sample 106.

A flow outlet 124 is fluidly coupled to the core sample assembly 110 (and thus the core sample 106) through the pressurized container 102. The flow outlet 124 is also fluidly coupled to a downstream fluid reservoir 118. Downstream fluid reservoir 118 also includes or is in fluid communication with a downstream fluid pump 122 that is operable to circulate a test fluid 120 (which can be the same fluid as test fluid 130) through the flow outlet 124 from the core sample assembly 110 to, e.g., controllably change or maintain a pressure at a second end (e.g., a downstream end) of the core sample 106. In the present disclosure, one or more of the described fluid pumps can be, for example, a high accuracy pressure gas pump.

The core sample test system 100 includes fluid sensors 136, 138, and 140, as well as fluid pumps 116, 122, and 132. Each of the fluid sensors 138-140 can be operable to measure a characteristic, such as pressure, temperature, or a combination thereof, of a fluid flow through the core sample within the test system 100 and the fluid sensor 136 can be operable to measure a characteristic, such as pressure, temperature, or a combination thereof, of a fluid applied to, but not in communication with, the core sample within the test system 100. Each of the fluid pumps among 116, 122, and 132 can be operable to measure a characteristic, such as the volume, volume change, the volume change with time (e.g., flow rate), or a combination thereof, of a fluid flow within the core sample test system 100. For example, fluid sensor 136 is positioned and operable to measure a characteristic (e.g., pressure) of pressurized fluid 114, which in turn can be identical to or substantially the same as a confining pressure of volume 104. Fluid sensor 138 is positioned and operable to measure a characteristic (e.g., pressure) of test fluid 120, which in turn can be identical to or substantially the same as a flow outlet pressure of the flow outlet 124. Fluid sensor 140 is positioned and operable to measure a characteristic (e.g., pressure) of test fluid 130, which in turn can be identical to or substantially the same as a flow inlet pressure of the flow inlet 134. Each of the fluid sensors 136-140 can be, for example, a high accuracy, high precision sensor capable of measuring both pressure and temperature simultaneously.

As shown in FIG. 1, a control system (or controller) 142 is communicably coupled to components of the system 100. Control system 142, in some aspects, can be a proportional, integral, derivative (PID) controller. For example, as shown, the control system 142 is communicably coupled to the fluid pumps 116, 122, and 132 to control operation (e.g., speed, on/off) of each fluid pump individually. Control of fluid pumps 116, 122, and 132 can be through control lines 144a, 144b, and 144f, respectively. Control system 142 is also communicably coupled to the fluid sensors 136, 138, and 140 through control lines 144c, 144d, and 144e, respectively, to receive measurements (e.g., pressure, temperature, flow rate, or a combination thereof). In some aspects, control system 142 is a microprocessor-based system that includes one or more hardware processors, one or more memory modules that store executable instructions (e.g., in MATLAB code), and one or more communication or network interfaces to allow communication between the control system 142 and the fluid pumps and fluid sensors shown in FIG. 1. Although not shown, additional components, such as valves (e.g., modulating or shut-off), power supplies, and/or pump motors can also be included within the system 100 for operation.

Notably, operation of the core sample test system 100 in this example implementation of FIG. 1 avoids a “point by point measurement” approach, in which one test run of the system 100 would only provide one permeability data point at the pore pressure and confining pressure and all test runs are lengthy processes by themselves. In addition, for each measurement in a point-by-point measurement approach requires a lengthy period of preparation process. The use of the point-by-point measurement (in other words, a conventional approach) is often due to the requirement of data analysis that inside a core sample there must be a small pressure difference such that gas properties, such as the compressibility, density, and viscosity, can be approximated as constants.

In addition, operation of the core sample test system 100 in this example implementation of FIG. 1 avoids single or multiple transducers installed on the flow path of the core sample (i.e., between the two ends of a core sample). For instance, as shown, the core sample assembly 110 is exclusive of any pressure or temperature transducer. The connection between the transducers and the core sample can require high dexterity to mount and can be a potential weak point to cause leaks between the confining fluid and the pore (or test) fluid.

In addition, as described in more detail herein, operation of the core sample test system 100 utilizes a steady state method, e.g., the pressure at a particular location in the core sample test system 100 does not appreciably change with time once in steady state. Also, by using, e.g., a relatively large pressure difference across core sample 106 (e.g., 1500 psi) as compared to that of conventional steady state systems (e.g., 10s of psi) allows for the steady state operation of core sample test system 100 and associated methods.

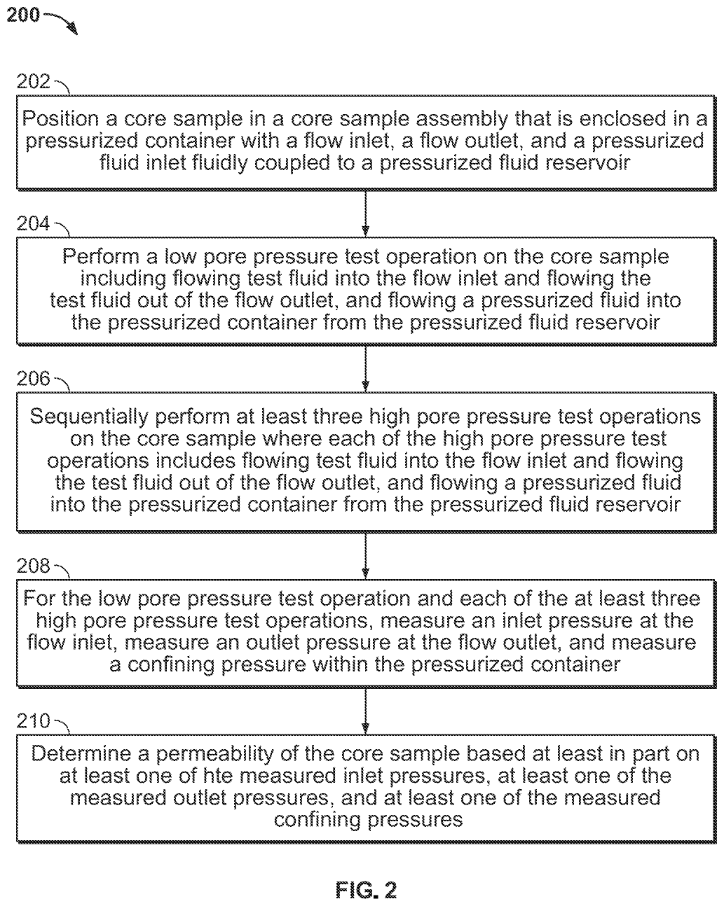

FIG. 2 is a flowchart of an example method 200 for measuring permeability of a core sample. The method 200 can be performed with or by the example implementation of the core sample test system 100 of FIG. 1. Generally, the method 200 includes four or more large-pressure-differential steady state test operations. For example, method 200 can be implemented and include a low pore pressure operational run and at least three high pore pressure operational runs of steady state experiments with different pressure differences between the upstream fluid reservoir 128 and downstream fluid reservoir 118 and/or different confining pressures in the volume 104. In such steady-state measurement tests, the upstream and downstream pressures (in flow inlet 134 and flow outlet 124, respectively) can be kept constant (or substantially constant) at selected pressures with the control system 142.

A core sample is positioned in a core sample assembly (or sample stack) that is enclosed in a pressurized container with a flow inlet, a flow outlet, and a pressurized fluid inlet fluidly coupled to a pressurized fluid reservoir (step 202). For example, as shown in FIG. 1, the core sample 106 is placed in the core sample assembly 110 and enclosed by sleeve 108 around a radial surface of (in this example) the cylindrically-shaped sample 106. In this example, therefore, axial faces 105a and 105b of the core sample 106 are exposed to the flow inlet 134 and the flow outlet 124, respectively. The core sample assembly 110, as shown, is positioned in the volume 104 of the pressurized container 102 and thus exposed to the pressure of the pressurized fluid 114 at the pressurized fluid inlet 126. In some aspects, it is ensured that there is no detected leakage from all the connections to the sample 106 within the pressurized container 102 such that there is no fluid exchange between the pressurized fluid 114 and the fluid flowing through the sample from 130 to 120.

A low pore pressure steady state test operation with a large pore pressure differential on the core sample includes flowing a test fluid into the flow inlet, flowing the test fluid out of the flow outlet, and flowing a pressurized fluid into the pressurized container from the pressurized fluid reservoir (step 204). For example, the low pore pressure test operation includes operating the upstream fluid pump 132, the downstream fluid pump 122, and the pressurized fluid pump 116 to provide or maintain a particular upstream pressure at the axial face 105a, a particular downstream pressure at the axial face 105b, and a particular confining pressure in the volume 104. In an example, for the low pore pressure test operation, the upstream pressure is 2015 psi, the downstream pressure is 15 psi, and the confining pressure is 2515. In this example, the difference between the confining pressure and the upstream pressure is 500 psi and the difference between the confining pressure and the downstream pressure is 2500 psi. The high pressure differential between the upstream pressure and the downstream pressure result in complicated non-linear flow interactions as the rock properties and fluid properties change along the flow path of the core sample.

At least three high pressure test operations with large pore pressure differentials are sequentially performed on the core sample that include flowing a test fluid into the flow inlet, flowing the test fluid out of the flow outlet, and flowing a pressurized fluid into the pressurized container from the pressurized fluid reservoir (step 206). For example, each high pore pressure test operation includes operation of the upstream fluid pump 132, the downstream fluid pump 122, and the pressurized fluid pump 116 to provide or maintain a particular upstream pressure at the axial face 105a, a particular downstream pressure at the axial face 105b, and a particular confining pressure in the volume 104.

The upstream pressure, the downstream pressure, and the confining pressure in each of the at least three high pore pressure test operations can be greater than the pressures in the low pore pressure test operation. The effect of diffusion can be minimized for in the permeability measurements by using the high pressures in the test operations.

The three high pore pressure steady state test operations with large pore pressure differentials can be viewed to include two pairs of operations: a first pair of test operations can include a first test run and a third test run, while a second pair of test operations can include the first test run and a second test run. In other example implementations, the pairing of test runs can be changed e.g., a first pair of test operations can include the first test run and the second test run, while a second pair of test operations can include the first test run and the third test run).

In this example implementation of method 200, the high pore pressure test runs in the first pair can have the same (or substantially similar) upstream pressures (at the axial face 105a) and the same (or substantially similar) downstream pressures (at the axial face 105b) but different confining pressures in the volume 104. The second pair of the high pore pressure test runs can have the same confining pressures in volume 104 and the same fluid pressures in one reservoir (i.e., at one axial face of the core sample 106), such as the upstream flow reservoir 128 or downstream flow reservoir 118, but different fluid pressures in the other reservoir (i.e., at the other axial face of the core sample 106).

In some aspects of method 200, to minimize the effects of hysteresis, a sequence of the low pore pressure test runs and the three high pressure test runs can be conducted so that an effective stress of the core sample 106 can keep constant/increasing or constant/decreasing during the three test runs.

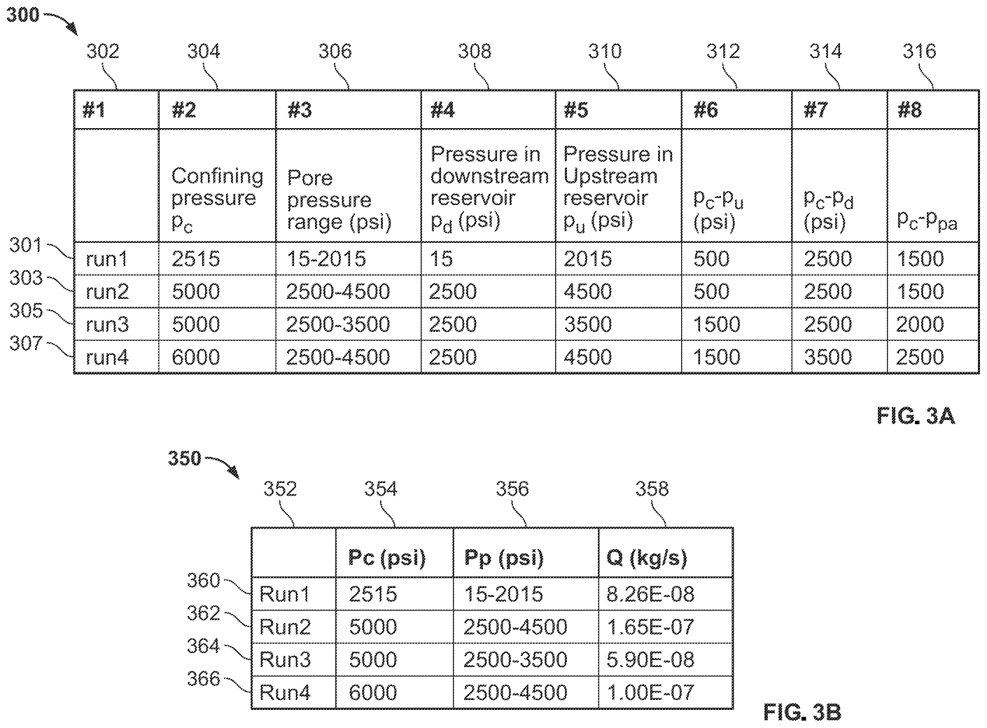

For the low pore pressure test operation and each of the at least three high pore pressure test operations, an inlet pressure at the flow inlet, an outlet pressure at the flow outlet, and a confining pressure within the pressurized container are measured (step 208). These measurements can be performed until the experiment reaches a steady state. For example, as shown in Table 300 in FIG. 3A, measurements can be taken by the fluid sensors 136, 138, and 140 to measure a characteristic (such as pressure, temperature, or flow rate or a combination thereof) of a particular fluid in the core sample test system 100. For example, flow sensor 140 measures, in this example, a pressure in the flow inlet 134 (and thus at the axial face 105a). Flow sensor 138 measures, in this example, a pressure in the flow outlet 124 (and thus at the axial face 105b). Flow sensor 136 measures, in this example, a pressure in the pressurized fluid inlet 126 (and thus in volume 104).

Table 300 shows, in columns 302-316 left to right: Test Run Number (302); confining pressure (304) (pressure in volume 104), p_{c}; “pore” pressure range (306) (e.g., a range between downstream pressure at axial face 105b and upstream pressure at axial face 105a); a pressure (308) in upstream flow reservoir 128, pᵤ; a pressure (310) in downstream flow reservoir 118, pa; a difference in confining pressure and upstream pressure (312); a difference in confining pressure and downstream pressure (314); and an average of the differences (316) shown in the previous two columns. The measured and determined pressure values of the first, second, third and fourth test runs are shown in rows 301, 303, 305 and 307, respectively. In this example, the first run 301 is the low pore pressure test run where the pore pressure is less than, for example, 2500 psi and both the compaction and the Knudsen diffusion affect the measurement results, and the second through fourth runs 303, 305, 307 are the high pore pressure test runs where the pore pressures are, for example, larger than 2500 psi where the effect of Knudsen diffusion is greatly reduced.

In this example of method 200, therefore, a first pair of high pore pressure test runs, test run 2 and test run 4, share an upstream pressure, pᵤ, of 4,500 psi and a downstream pressure, p_{d}, of 2,500 psi, but have different confining pressures (p_{c}) of 5000 psi and 6000 psi, respectively. A second pair of high pore pressure test runs, test run 2 and test run 3, share the same downstream pressure, p_{d}, of 2,500 psi; the same confining pressure, p_{c}, of 5000 psi, but different upstream pressure, pᵤ, 4500 psi and 3500 psi, respectively. In some implementations, test run 2 and test run 3 share the same upstream pressure and have different downstream pressures. In this example, the test run order (test run 1, then test run 2, then test run 3, then test run 4) is chosen to ensure that the effective stress keeps constant/increasing from one test to the next, as suggested by the average of the pressure differences (316) in Table 300. Further, for a steady-state flow test run (such as test runs 1, 2, 3, and 4), the gas pressure at the inlet of the core sample 106 (e.g., within the flow inlet 134 at axial face 105a) is the same as the pressure in the upstream fluid reservoir 128, pᵤ, and is kept constant or substantially constant. The gas pressure at the outlet of the core sample 106 (e.g., within the flow outlet 124 at axial face 105b) is the same as the pressure in the downstream fluid reservoir 118, p_{d}, and is kept constant or substantially constant. In some aspects of method 200, the tubing connected to the inlet and outlet of a core sample is assumed to produce no resistance to the flow (or its permeability is assumed to be infinitely large).

For each test, pre-specified or desired constant gas pressures are achieved and maintained in the upstream and downstream fluid reservoirs 128 and 118, respectively, as well as the pressurized fluid reservoir 112. Then, the test fluid 130 flows from the flow inlet 134 to the flow outlet 124, because the inlet gas pressure, pᵤ, is higher than the outlet gas pressure, p_{d}. After reaching the steady state, the pressure within the sample 106 does not change with time. After the steady state flow process is achieved along the rock sample, a mass flow rate, Q, of the test fluid 130 (and therefore test fluid 120) is independent of time, e.g., the flow rate remains constant. In some aspects, when an apparent average permeability (as determined in step 210) does not change more than 5% within a particular time duration (e.g., four hours or another selected time duration), the steady state flow condition is achieved.

Method 200 can continue at step 210, which includes determining a permeability of the core sample based at least in part on at least one of the measured inlet pressures, at least one of the measured outlet pressures, and at least one of the measured confining pressures. For example, in some aspects, determination of the apparent permeability of the core sample 106 is based at least in part on an average of the upstream and downstream flow rates in mass and Darcy's law, which can be used to calculate a permeability (e.g., an apparent average permeability) of the core sample 106.

In some aspects, other characteristics of the core sample 106 can be determined prior to determination of the apparent average permeability, such as a pressure dependence coefficient, α, a poroelastic coefficient, β, of the core sample 106, and many diffusion correction factors, f_{c} along the flow path within the sample.

For the low pore pressure rest run, (e.g., run 1 in Table 300), a diffusion correction factor for the permeability can be determined to account for the Knudsen effect. The Knudsen effect occurs, for example, when the mean free path of the gas molecules is larger than the pore diameter (e.g., pore throat size). The Knudsen effect can be important even at high pore pressures when the pore throat in the core sample is extremely small (e.g., dozens of nanometers). Using the diffusion correction factor the Knudsen diffusion can be considered for both low pore pressure and high pore pressure test runs, e.g., test runs 1 through 4 of Table 300.

The apparent permeability, ǩ, is the compaction-dependent permeability, k, at a location x, multiplied by the correction factors f_{c} due to the Knudsen diffusion effect, ǩₓ=f_{c}k. Multiple correction factors f_{c} are used due to the changing rock and fluid properties along the flow path.

The mass flow rate Q, under steady-state flow conditions, can be expressed as shown in equation (1) at any point x for the length L and the cross-section area A of the sample, the apparent permeability ǩ, the gas density ρ, the gas viscosity μ, and the pore pressure p.

$$Q = - A \frac{\check{k} ρ}{μ} \frac{\partial p}{\partial x} \qquad ( 1 )$$

Because the permeability is an exponential function of effective stress, the apparent permeability, ǩ, is related to the compaction-dependent permeability k at any location x by:

$$\check{k} = {f}_{c} k = {f}_{c} {k}_{0} exp [ - α ( {p}_{c} - β p ) ] . \qquad ( 2 )$$

For low pore pressure tests, f_{c}>>1, or alternatively, ǩ>>k. For the high pore pressure tests, there is little or no Knudsen diffusion, e.g., f_{c}≈1 and ǩ≈k at the points along the flow path.

The correction factor f_{c} is further related to the average diameter of the pores, r, at the location x, which changes with effective stress (r₀ denotes the pore radius when the effective stress is zero). Since the permeability is proportional to the square of the average radius of pores,

$${( \frac{r}{{r}_{0}} )}^{2} = \frac{k}{{k}_{o}} = exp [ - α ( {p}_{c} - β p ) ] \qquad ( 3 )$$

Note that f_{c}, the correction factor, can be expressed as

$${f}_{c} = ( 1 + \frac{{α}_{0}}{1 + \frac{\tilde{A}}{{N}^{B}}} ) ( 1 + \frac{4 N}{1 - bN} ) , \qquad ( 4 )$$

$$N = \frac{λ}{r} , \qquad ( 5 )$$

$$λ = \frac{{k}_{B} T}{\sqrt{2} π {d}_{c}^{2} p} , \qquad ( 6 )$$

where λ is the mean free path length, k_{B} is the Boltzmann constant (1.3805 9 10-23 J/K), T is the temperature, in K, p is the pore pressure, in Pa, d_{c} (m) is the collision diameter for the working fluid (e.g., d_{c}=0.42 nm for methane and d_{c}=0.358 nm for N₂); r is the effective radius of pore throats in the core sample; b is a constant and equal to −1, α₀=64/(15 acos (−1)), Ã=0.1780 and B=0.4348.

Integrating equation (1) from the inlet (x=0) to the outlet x=L yields

$${\int }_{0}^{L} Qdx = - A {\int }_{{p}_{u}}^{{p}_{d}} \frac{\check{k} ρ}{μ} dp . \qquad ( 7 )$$

Since Q is a constant at steady state, this simplifies to

$$\frac{QL}{A} = {\int }_{{p}_{d}}^{{p}_{u}} \frac{\check{k} ρ}{μ} dp \qquad ( 8 )$$

Let k₀ represent the permeability when the effective stress equals zero and let k_{d} and kᵤ represent the permeabilities of the rock when Knudsen diffusion is absent and pore pressures are p_{d} and pᵤ, respectively. Then relationships between the apparent permeability ǩ and pore pressure p and confining stress or pressure p_{c} are given as follows:

$$k = {k}_{0} exp [ - α ( {p}_{c} - β p ) ] for any x with pore pressure p , \qquad ( 9 )$$

$${k}_{d} = {k}_{0} exp [ - α ( {p}_{c} - β {p}_{d} ) ] for x = L , \qquad ( 10 )$$

$$\check{k} = {f}_{c} {k}_{0} exp [ - α ( {p}_{c} - β p ) ] for 0 < x < L . \qquad ( 11 )$$

Combining equations (9) and (10) gives:

$$\check{k} = {f}_{c} {k}_{d} exp [ - α β ( {p}_{d} - p ) ] for 0 < x < L \qquad ( 12 )$$

Plugging ǩ into Eq. (8) yields

$$\frac{Q L}{A} = {k}_{d} {\int }_{{p}_{d}}^{{p}_{u}} \frac{{f}_{c} exp [ - α β ( {p}_{d} - p ) ] ρ dp}{μ} \qquad ( 13 )$$

The first pair of high pore pressure test runs (e.g., test runs 2 and 4) have the same inlet pressure pᵤ and outlet pressure pa but different confining pressure p_{c}, enabling a solution for a ratio of the mass flow rates, Q2/Q4, as shown below. Subscript numbers associated with variables indicate the run number. Equation (13) is written for both runs and Eq. (10) is substituted for k_{d}.

$$\frac{{Q}_{2} L}{A} = {k}_{0} exp [ - α ( {p}_{c 2} - β {p}_{d} ) ] {\int }_{{p}_{d}}^{{p}_{u}} \frac{{f}_{c} exp [ - a β ( {p}_{d} - p ) ] ρ dp}{μ} \qquad ( 14 )$$

$$\frac{{Q}_{4} L}{A} = {k}_{0} exp [ - α ( {p}_{c 4} - β {p}_{d} ) ] {\int }_{{p}_{d}}^{{p}_{u}} \frac{{f}_{c} exp [ - a β ( {p}_{d} - p ) ] ρ dp}{μ} \qquad ( 15 )$$

As both of the test runs are at high pore pressure, the f_{c} on all points for both test runs are close to 1, dividing equation (14) by equation (15) gives:

$$\frac{{Q}_{2}}{{Q}_{4}} = exp [ - α ( {p}_{c 2} - {p}_{c 4} ) ] . \qquad ( 16 )$$

Equation (16) can be solved for the pressure dependent coefficient α:

$$α = \frac{ln ( \frac{{Q}_{2}}{{Q}_{4}} )}{( {p}_{c 4} - {p}_{c 2} )} . \qquad ( 17 )$$

Inserting the expression for f_{c} into Eq. (13) for test run 1 gives:

$$\frac{{Q}_{1} L}{A} = {k}_{0} {\int }_{{P}_{d 1}}^{{p}_{u 1}} ( 1 + \frac{{α}_{0}}{1 + \frac{\tilde{A}}{{( \frac{\frac{{k}_{B} T}{\sqrt{2} π {d}_{c}^{2} p}}{{r}_{0} \sqrt{exp [ α ( {p}_{c 1} - β p ) ]}} )}^{B}}} ) * ( 1 + \frac{4}{( \frac{1}{\frac{\frac{{k}_{B} T}{\sqrt{2} π {d}_{c}^{2} p}}{{r}_{0} \sqrt{exp [ α ( {p}_{c 1} - β p ) ]}}} ) - b} ) \frac{exp [ - α ( {p}_{c 1} - β p ) ] ρ}{μ} dp \qquad ( 18 )$$

$$\frac{{Q}_{2} L}{A} = {k}_{0} {\int }_{{P}_{d 2}}^{{p}_{u 2}} ( 1 + \frac{{α}_{0}}{1 + \frac{\tilde{A}}{{( \frac{\frac{{k}_{B} T}{\sqrt{2} π {d}_{c}^{2} p}}{{r}_{0} \sqrt{exp [ α ( {p}_{c 2} - β p ) ]}} )}^{B}}} ) * ( 1 + \frac{4}{( \frac{1}{\frac{\frac{{k}_{B} T}{\sqrt{2} π {d}_{c}^{2} p}}{{r}_{0} \sqrt{exp [ α ( {p}_{c 2} - β p ) ]}}} ) - b} ) \frac{exp [ - α ( {p}_{c 2} - β p ) ] ρ}{μ} dp \qquad ( 19 )$$

$$\frac{{Q}_{3} L}{A} = {k}_{0} {\int }_{{P}_{d 3}}^{{p}_{u 3}} ( 1 + \frac{{α}_{0}}{1 + \frac{\tilde{A}}{{( \frac{\frac{{k}_{B} T}{\sqrt{2} π {d}_{c}^{2} p}}{{r}_{0} \sqrt{exp [ α ( {p}_{c 3} - β p ) ]}} )}^{B}}} ) * ( 1 + \frac{4}{( \frac{1}{\frac{\frac{{k}_{B} T}{\sqrt{2} π {d}_{c}^{2} p}}{{r}_{0} \sqrt{exp [ α ( {p}_{c 3} - β p ) ]}}} ) - b} ) \frac{exp [ - α ( {p}_{c 3} - β p ) ] ρ}{μ} dp \qquad ( 20 )$$

Where subscript 1 refers to test run 1. Similar equations can be obtained for runs 2 and 3 by changing the subscript to 2 and 3 respectively from equation 18 to generate equations 19 and 20. There are three unknowns k₀, β, and r₀ in these three equations. The values of the unknowns can be obtained (e.g., numerically solved) based on the three equations. Based on these parameter values and the related expressions associated with the parameters, the apparent permeability value for a given effective stress and a pore pressure can be determined. Since runs 2, 3, 4 are carried with high pore pressure, it is also possible to assume the f_{c}=1 for all these tests and use methods of U.S. Pat. No. 11,680,887, which is hereby incorporated by reference in its entirety, to resolve α, k₀, β, and use equation 18 to resolve r₀.

FIG. 3B shows Table 350 including results from an example implementation of method 200. In this example, the core sample is one inch in diameter and two inches long. Nitrogen (N2) is used as the working fluid. Column 352 shows the test run number, column 354 shows the confining pressure, column 356 shows the pore pressure, and column 358 shows the mass flow rate. The rows 360-366 show the test runs with row 360 corresponding to the low pore pressure test run, and rows 362-366 corresponding to the high pore pressure test runs. Using the Table 350 with equation 17 and equations 18-20, the stress dependence

$$α = \frac{ln ( \frac{{Q}_{2}}{{Q}_{4}} )}{{pc}_{4} - {pc}_{2}} ,$$

is estimated to be 5E-4 psi-1; and other parameters can be determined as follows, r₀ to be 100 nm, k₀ to be 300 E-21 m², and β to be 0.9.

Method 200 can include additional steps as well. For example, once the permeability (e.g., stress-dependent apparent permeability) is determined for the core sample 106, a hydrocarbon production rate from a reservoir from which the sample 106 came can be predicted. Note that when simulating gas production (e.g., from a shale formation) with Darcy's law, apparent permeability can yield more accurate results than compaction-dependent permeability alone. For example, during the hydrocarbon production from an unconventional reservoir, the pore pressure decreases with time and thus effective stress will change with time as well. In that case, permeability will be a function of both time and location as a result of stress alteration during the production. The stress-dependent permeability determined in step 208 can be used as an input into a reservoir simulator to more accurately predict the hydrocarbon production because the measured stress dependency captures the permeability evolution during the production. The determined and predicted parameters of the core sample 106 can be graphically represented on a GUI of the control system 142.

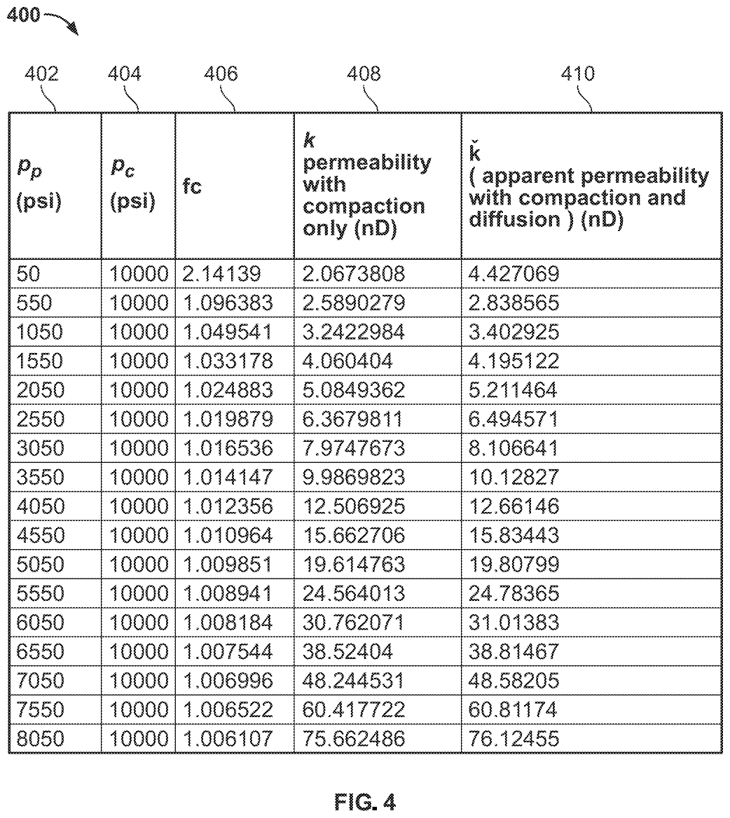

FIG. 4 shows Table 400 with permeability values for compaction only and for compaction with diffusion. Column 402 is the pore pressure, column 404 is the confining pressure, column 406 is the correction factor, column 408 is the compaction-dependent permeability, column 410 is the apparent permeability with compaction and diffusion. Particularly for low pore pressures, the effects of diffusion significantly increase the apparent permeability as compared with the compaction-only permeability. For example, Knudsen diffusion can cause the gas to move faster resulting in a high gas production than estimated without considering Knudsen diffusion. Even at high pore pressures, the diffusion effects increase the apparent permeability.

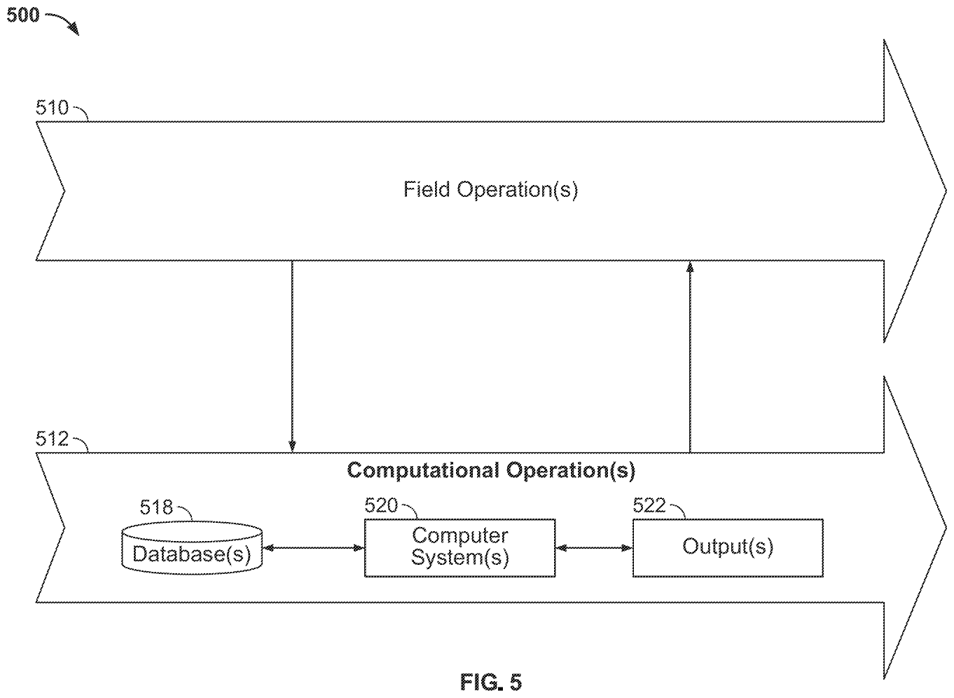

FIG. 5 illustrates hydrocarbon production operations 500 that include both one or more field operations 510 and one or more computational operations 512, which exchange information and control exploration for the production of hydrocarbons. In some implementations, outputs of techniques of the present disclosure (e.g., the method 200) can be performed before, during, or in combination with the hydrocarbon production operations 500, specifically, for example, either as field operations 510 or computational operations 512, or both. For example, the method 200 collects data during field operations, processes the data in computational operations, and can determine locations to perform additional field operations.

Examples of field operations 510 include forming/drilling a wellbore, hydraulic fracturing, producing through the wellbore, injecting fluids (such as water) through the wellbore, to name a few. In some implementations, methods of the present disclosure can trigger or control the field operations 510. For example, the methods of the present disclosure can generate data from hardware/software including sensors and physical data gathering equipment (e.g., seismic sensors, well logging tools, flow meters, and temperature and pressure sensors). The methods of the present disclosure can include transmitting the data from the hardware/software to the field operations 510 and responsively triggering the field operations 510 including, for example, generating plans and signals that provide feedback to and control physical components of the field operations 510. Alternatively, or in addition, the field operations 510 can trigger the methods of the present disclosure. For example, implementing physical components (including, for example, hardware, such as sensors) deployed in the field operations 510 can generate plans and signals that can be provided as input or feedback (or both) to the methods of the present disclosure.

Examples of computational operations 512 include one or more computer systems 520 that include one or more processors and computer-readable media (e.g., non-transitory computer-readable media) operatively coupled to the one or more processors to execute computer operations to perform the methods of the present disclosure. The computational operations 512 can be implemented using one or more databases 518, which store data received from the field operations 510 and/or generated internally within the computational operations 512 (e.g., by implementing the methods of the present disclosure) or both. For example, the one or more computer systems 520 process inputs from the field operations 510 to assess conditions in the physical world, the outputs of which are stored in the databases 518. For example, seismic sensors of the field operations 510 can be used to perform a seismic survey to map subterranean features, such as facies and faults. In performing a seismic survey, seismic sources (e.g., seismic vibrators or explosions) generate seismic waves that propagate in the earth and seismic receivers (e.g., geophones) measure reflections generated as the seismic waves interact with boundaries between layers of a subsurface formation. The source and received signals are provided to the computational operations 512 where they are stored in the databases 518 and analyzed by the one or more computer systems 520.

In some implementations, one or more outputs 522 generated by the one or more computer systems 520 can be provided as feedback/input to the field operations 510 (either as direct input or stored in the databases 518). The field operations 510 can use the feedback/input to control physical components used to perform the field operations 510 in the real world.

For example, the computational operations 512 can process the seismic data to generate three-dimensional (3D) maps of the subsurface formation. The computational operations 512 can use these 3D maps to provide plans for locating and drilling exploratory wells. In some operations, the exploratory wells are drilled using logging-while-drilling (LWD) techniques which incorporate logging tools into the drill string. LWD techniques can enable the computational operations 512 to process new information about the formation and control the drilling to adjust to the observed conditions in real-time.

The one or more computer systems 520 can update the 3D maps of the subsurface formation as information from one exploration well is received and the computational operations 512 can adjust the location of the next exploration well based on the updated 3D maps. Similarly, the data received from production operations can be used by the computational operations 512 to control components of the production operations. For example, production well and pipeline data can be analyzed to predict slugging in pipelines leading to a refinery and the computational operations 512 can control machine operated valves upstream of the refinery to reduce the likelihood of plant disruptions that run the risk of taking the plant offline.

In some implementations of the computational operations 512, customized user interfaces can present intermediate or final results of the above-described processes to a user. Information can be presented in one or more textual, tabular, or graphical formats, such as through a dashboard. The information can be presented at one or more on-site locations (such as at an oil well or other facility), on the Internet (such as on a webpage), on a mobile application (or app), or at a central processing facility.

The presented information can include feedback, such as changes in parameters or processing inputs, that the user can select to improve a production environment, such as in the exploration, production, and/or testing of petrochemical processes or facilities. For example, the feedback can include parameters that, when selected by the user, can cause a change to, or an improvement in, drilling parameters (including drill bit speed and direction) or overall production of a gas or oil well. The feedback, when implemented by the user, can improve the speed and accuracy of calculations, streamline processes, improve models, and solve problems related to efficiency, performance, safety, reliability, costs, downtime, and the need for human interaction.

In some implementations, the feedback can be implemented in real-time, such as to provide an immediate or near-immediate change in operations or in a model. The term real-time (or similar terms as understood by one of ordinary skill in the art) means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second(s), or less than 5 s. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit the data.

Events can include readings or measurements captured by downhole equipment such as sensors, pumps, bottom hole assemblies, or other equipment. The readings or measurements can be analyzed at the surface, such as by using applications that can include modeling applications and machine learning. The analysis can be used to generate changes to settings of downhole equipment, such as drilling equipment. In some implementations, values of parameters or other variables that are determined can be used automatically (such as through using rules) to implement changes in oil or gas well exploration, production/drilling, or testing. For example, outputs of the present disclosure can be used as inputs to other equipment and/or systems at a facility. This can be especially useful for systems or various pieces of equipment that are located several meters or several miles apart or are located in different countries or other jurisdictions.

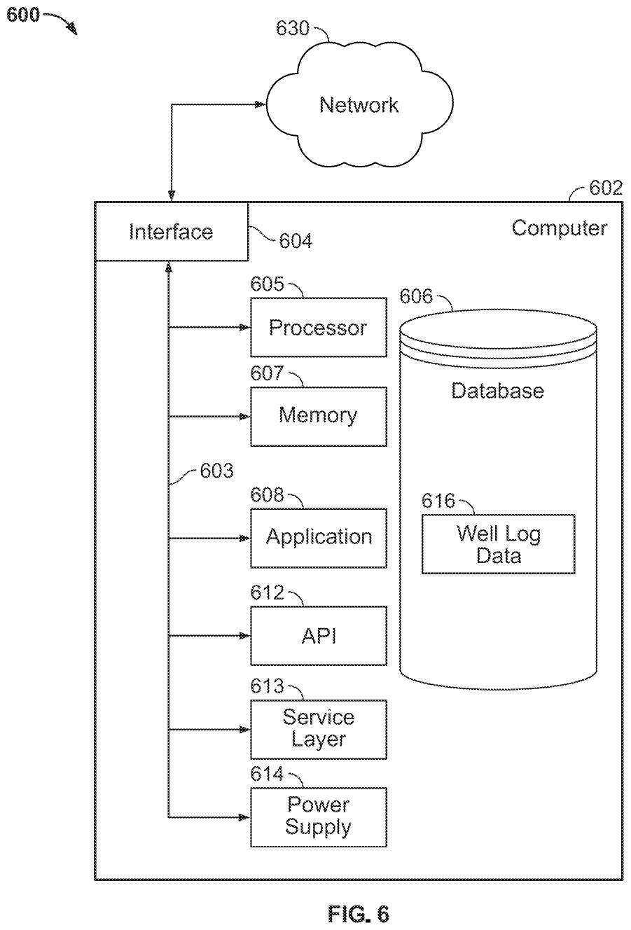

FIG. 6 is a block diagram of an example computer system 600 used to provide computational functionalities associated with described algorithms, methods, functions, processes, flows, and procedures described in the present disclosure, according to some implementations of the present disclosure. The illustrated computer 602 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 602 can include input devices such as keypads, keyboards, and touch screens that can accept user information. Also, the computer 602 can include output devices that can convey information associated with the operation of the computer 602. The information can include digital data, visual data, audio information, or a combination of information. The information can be presented in a graphical user interface (UI) (or GUI).

The computer 602 can serve in a role as a client, a network component, a server, a database, a persistency, or components of a computer system for performing the subject matter described in the present disclosure. The illustrated computer 602 is communicably coupled with a network 630. In some implementations, one or more components of the computer 602 can be configured to operate within different environments, including cloud-computing-based environments, local environments, global environments, and combinations of environments.

At a high level, the computer 602 is an electronic computing device operable to receive, transmit, process, store, and manage data and information associated with the described subject matter. According to some implementations, the computer 602 can also include, or be communicably coupled with, an application server, an email server, a web server, a caching server, a streaming data server, or a combination of servers.

The computer 602 can receive requests over network 630 from a client application (for example, executing on another computer 602). The computer 602 can respond to the received requests by processing the received requests using software applications. Requests can also be sent to the computer 602 from internal users (for example, from a command console), external (or third) parties, automated applications, entities, individuals, systems, and computers.

Each of the components of the computer 602 can communicate using a system bus 603. In some implementations, any or all of the components of the computer 602, including hardware or software components, can interface with each other or the interface 604 (or a combination of both), over the system bus 603. Interfaces can use an application programming interface (API) 612, a service layer 613, or a combination of the API 612 and service layer 613. The API 612 can include specifications for routines, data structures, and object classes. The API 612 can be either computer-language independent or dependent. The API 612 can refer to a complete interface, a single function, or a set of APIs.

The service layer 613 can provide software services to the computer 602 and other components (whether illustrated or not) that are communicably coupled to the computer 602. The functionality of the computer 602 can be accessible for all service consumers using this service layer. Software services, such as those provided by the service layer 613, can provide reusable, defined functionalities through a defined interface. For example, the interface can be software written in JAVA, C++, or a language providing data in extensible markup language (XML) format. While illustrated as an integrated component of the computer 602, in alternative implementations, the API 612 or the service layer 613 can be stand-alone components in relation to other components of the computer 602 and other components communicably coupled to the computer 602. Moreover, any or all parts of the API 612 or the service layer 613 can be implemented as child or sub-modules of another software module, enterprise application, or hardware module without departing from the scope of the present disclosure.

The computer 602 includes an interface 604. Although illustrated as a single interface 604 in FIG. 6, two or more interfaces 604 can be used according to particular needs, desires, or particular implementations of the computer 602 and the described functionality. The interface 604 can be used by the computer 602 for communicating with other systems that are connected to the network 630 (whether illustrated or not) in a distributed environment. Generally, the interface 604 can include, or be implemented using, logic encoded in software or hardware (or a combination of software and hardware) operable to communicate with the network 630. More specifically, the interface 604 can include software supporting one or more communication protocols associated with communications. As such, the network 630 or the interface's hardware can be operable to communicate physical signals within and outside of the illustrated computer 602.

The computer 602 includes a processor 605. Although illustrated as a single processor 605 in FIG. 6, two or more processors 605 can be used according to particular needs, desires, or particular implementations of the computer 602 and the described functionality. Generally, the processor 605 can execute instructions and can manipulate data to perform the operations of the computer 602, including operations using algorithms, methods, functions, processes, flows, and procedures as described in the present disclosure.

The computer 602 also includes a database 606 that can hold data for the computer 602 and other components connected to the network 630 (whether illustrated or not). For example, database 606 can be an in-memory, conventional, or a database storing data consistent with the present disclosure. For example, database 606 can hold well log data 616. In some implementations, database 606 can be a combination of two or more different database types (for example, hybrid in-memory and conventional databases) according to particular needs, desires, or particular implementations of the computer 602 and the described functionality. Although illustrated as a single database 606 in FIG. 6, two or more databases (of the same, different, or combination of types) can be used according to particular needs, desires, or particular implementations of the computer 602 and the described functionality. While database 606 is illustrated as an internal component of the computer 602, in alternative implementations, database 606 can be external to the computer 602.

The computer 602 also includes a memory 607 that can hold data for the computer 602 or a combination of components connected to the network 630 (whether illustrated or not). Memory 607 can store any data consistent with the present disclosure. In some implementations, memory 607 can be a combination of two or more different types of memory (for example, a combination of semiconductor and magnetic storage) according to particular needs, desires, or particular implementations of the computer 602 and the described functionality. Although illustrated as a single memory 607 in FIG. 6, two or more memories 607 (of the same, different, or combination of types) can be used according to particular needs, desires, or particular implementations of the computer 602 and the described functionality. While memory 607 is illustrated as an internal component of the computer 602, in alternative implementations, memory 607 can be external to the computer 602.

The application 608 can be an algorithmic software engine providing functionality according to particular needs, desires, or particular implementations of the computer 602 and the described functionality. For example, application 608 can serve as one or more components, modules, or applications. Further, although illustrated as a single application 608, the application 608 can be implemented as multiple applications 608 on the computer 602. In addition, although illustrated as internal to the computer 602, in alternative implementations, the application 608 can be external to the computer 602.

The computer 602 can also include a power supply 614. The power supply 614 can include a rechargeable or non-rechargeable battery that can be configured to be either user- or non-user-replaceable. In some implementations, the power supply 614 can include power-conversion and management circuits, including recharging, standby, and power management functionalities. In some implementations, the power-supply 614 can include a power plug to allow the computer 602 to be plugged into a wall socket or a power source to, for example, power the computer 602 or recharge a rechargeable battery.

There can be any number of computers 602 associated with, or external to, a computer system containing computer 602, with each computer 602 communicating over network 630. Further, the terms “client,” “user,” and other appropriate terminology can be used interchangeably, as appropriate, without departing from the scope of the present disclosure. Moreover, the present disclosure contemplates that many users can use one computer 602 and one user can use multiple computers 602.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example LINUX, UNIX, WINDOWS, MAC OS, ANDROID, or IOS.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

A number of embodiments of these systems and methods have been described. Nevertheless, it will be understood that various modifications may be made without departing from the spirit and scope of this disclosure. Accordingly, other embodiments are within the scope of the following claims.

### EXAMPLES

In an example implementation, A method for measuring rock permeability incorporating diffusion effects, the method comprising positioning a core sample in a core sample assembly that is enclosed in a pressurized container comprising a flow inlet, a flow outlet, and a pressurized fluid inlet fluidly coupled to a pressurized fluid reservoir that comprises a pressurized fluid pump; performing a low pore pressure test operation on the core sample, the low pore pressure test operation comprising flowing a test fluid through the core sample between the flow inlet and the flow outlet with a first pore pressure, and maintaining a first confining pressure around the core sample by flowing a pressurized fluid into the pressurized container from the pressurized fluid reservoir; sequentially performing at least three high pore pressure test operations on the core sample, each of the at least three high pore pressure test operations comprising flowing the test fluid through the core sample between the flow inlet and the flow outlet, and maintaining a second pressure around the core sample by flowing a pressurized fluid into the pressurized container from the pressurized fluid reservoir, wherein the second confining pressure is at least 2500 psi and the second pore pressure is at least 1000 psi more than the first inlet pressure; for the low pore pressure test operation and each of the at least three high pore pressure test operations, measuring an inlet pressure at the flow inlet, measuring an outlet pressure at the flow outlet, and measuring a confining pressure within the pressurized container; determining a pressure dependent coefficient α, a permeability with zero effective stress k₀, a poroelastic coefficient β, of the core sample based at least in part on the measured inlet pressures, the measured outlet pressures, and the measured confining pressures of the three high pore pressure test operations; and determining a pore radius with zero effective stress r₀ of the core sample based on the determined α, k₀, and β, based at least in part on the measured inlet pressures, the measured outlet pressures, and the measured confining pressures of the low pore pressure test operation.

In an aspect combinable with the example implementation, the low pore pressure test operation includes operating a first pump to circulate the test fluid from a first reservoir, through the flow inlet, and to the core sample in the core sample assembly at the first inlet pressure; operating a second pump to circulate the test fluid from the core sample in the core sample assembly at a first outlet pressure, through the flow outlet, and to a second reservoir; and operating the pressurized fluid pump to circulate a pressurized fluid from the pressurized fluid reservoir to the pressurized container at the first confining pressure.

In another aspect combinable with any of the previous aspects, the at least three high pore pressure test operations include a first test operation that includes operating the first pump to circulate the test fluid from the first reservoir, through the flow inlet, and to the core sample in the core sample assembly at the second inlet pressure, operating a second pump to circulate the test fluid from the core sample in the core sample assembly at a second outlet pressure greater than the first outlet pressure, through the flow outlet, and to the second reservoir, and operating the pressurized fluid pump to circulate the pressurized fluid from the pressurized fluid reservoir to the pressurized container at the second confining pressure.

In another aspect combinable with any of the previous aspects, the at least three high pore pressure test operations comprises a second test operation subsequent to the first test operation, the second test operation includes operating the first pump to circulate the test fluid from the first reservoir, through the flow inlet, and to the core sample in the core sample assembly at a third inlet pressure different than the first and second inlet pressures, operating the second pump to circulate the test fluid from the core sample in the core sample assembly at the second outlet pressure, through the flow outlet, and to the second reservoir, and operating the pressurized fluid pump to circulate the pressurized fluid from the pressurized fluid reservoir to the pressurized container at the second confining pressure.

In another aspect combinable with any of the previous aspects, the at least three high pore pressure test operations comprises a third test operation subsequent to the first and second test operations, the third test operation includes operating the first pump to circulate the test fluid from the first reservoir, through the flow inlet, and to the core sample in the core sample assembly at the second inlet pressure, operating the second pump to circulate the test fluid from the core sample in the core sample assembly at the second outlet pressure, through the flow outlet, and to the second reservoir, and operating the pressurized fluid pump to circulate the pressurized fluid from the pressurized fluid reservoir to the pressurized container at a third confining pressure different than the first and second confining pressures.

In another aspect combinable with any of the previous aspects, the pressure dependence coefficient α of the core sample is determined based at least in part on the second inlet pressure, the second outlet pressure, and the second and third confining pressures.

In another aspect combinable with any of the previous aspects, determining the pressure dependence coefficient α of the core sample based at least in part on the second inlet pressure, the second outlet pressure, and the second and third confining pressures includes determining the pressure dependence coefficient α based on a first mass flow rate of the test fluid that is based on the second inlet pressure and the second outlet pressure, a second mass flow rate of the test fluid that is based on the second inlet pressure and the second outlet pressure, and a difference between the second and third confining pressures.

In another aspect combinable with any of the previous aspects, the poroelastic coefficient β of the core sample is determined based at least in part on the pressure dependence coefficient α.

In another aspect combinable with any of the previous aspects, determining the poroelastic coefficient β of the core sample based at least in part on the pressure dependence coefficient includes determining a product of the poroelastic coefficient β and the pressure dependence coefficient α based at least in part on a first mass flow rate of the test fluid that is based on the second inlet pressure and the second outlet pressure, a second mass flow rate of the test fluid that is based on the second inlet pressure and the second outlet pressure, the second and third inlet pressures, the second outlet pressure, and the second confining pressure; and determining the poroelastic coefficient β from the product of the poroelastic coefficient β and the pressure dependence coefficient α.

Another aspect combinable with any of the previous aspects includes determining the permeability of the core sample based at least in part on the poroelastic coefficient β and the pressure dependence coefficient α.

In another aspect combinable with any of the previous aspects, determining the permeability of the core sample based at least in part on the poroelastic coefficient β and the pressure dependence coefficient α includes determining the permeability of the core sample based at least in part on a first mass flow rate of the test fluid that is based on the first inlet pressure and the first outlet pressure; a cross-section area of the core sample; the poroelastic coefficient β; the pressure dependence coefficient α; a diffusion correction factor; a density of the test fluid; a viscosity of the test fluid; a distance between the flow inlet and the flow outlet; the first confining pressure; the first outlet pressure; and the first inlet pressure.

In another aspect combinable with any of the previous aspects, each of the measured inlet pressure, the measured outlet pressure, and the measured confining pressure is at a steady state condition.

In another example implementation, a system for determining a rock property includes a core sample assembly enclosed in a pressurized container that includes a flow inlet, a flow outlet, and a pressurized fluid inlet, the core sample assembly configured to secure a core sample; a pressurized fluid reservoir that includes a pressurized fluid pump, the pressurized fluid reservoir fluidly coupled to the pressurized fluid inlet; a first test fluid reservoir that includes a first pump, the first test fluid reservoir fluidly coupled to the flow inlet; a second test fluid reservoir that includes a second pump, the second test fluid reservoir fluidly coupled to the flow outlet; a plurality of fluid sensors, at least one fluid sensor positioned at or near each of the flow inlet, the flow outlet, and the pressurized fluid inlet; and a control system communicably coupled to the plurality of fluid sensors, the pressurized fluid pump, the first pump, and the second pump, the control system configured to perform operations including operating the pressurized fluid pump, the first pump, and the second pump to perform a low pore pressure test operation on the core sample and to sequentially perform at least three high pore pressure test operations on the core sample, each of the test operations including flowing a test fluid into the flow inlet from the first test fluid reservoir and flowing the test fluid out of the flow outlet into the second test fluid reservoir, and flowing a pressurized fluid into the pressurized container from the pressurized fluid reservoir; for the low pore pressure test operation and each of the at least three high pore pressure test operations, receiving, from the plurality of fluid sensors, measurements including an inlet pressure at the flow inlet, an outlet pressure at the flow outlet, and a confining pressure within the pressurized container; and determining a permeability of the core sample based at least in part on at least one of the measured inlet pressures, at least one of the measured outlet pressures, and at least one of the measured confining pressures.

In an aspect combinable with the example implementation, the low pore pressure test operation includes operating a first pump to circulate the test fluid from a first reservoir, through the flow inlet, and to the core sample in the core sample assembly at a first inlet pressure; operating a second pump to circulate the test fluid from the core sample in the core sample assembly at a first outlet pressure, through the flow outlet, and to a second reservoir; and operating the pressurized fluid pump to circulate a pressurized fluid from the pressurized fluid reservoir to the pressurized container at a first confining pressure.

In another aspect combinable with any of the previous aspects, the at least three high pore pressure test operations include a first test operation that includes operating the first pump to circulate the test fluid from the first reservoir, through the flow inlet, and to the core sample in the core sample assembly at a second inlet pressure greater than the first inlet pressure, operating a second pump to circulate the test fluid from the core sample in the core sample assembly at a second outlet pressure greater than the first outlet pressure, through the flow outlet, and to the second reservoir, and operating the pressurized fluid pump to circulate the pressurized fluid from the pressurized fluid reservoir to the pressurized container at a second confining pressure greater than the first confining pressure.

In another aspect combinable with any of the previous aspects, the at least three high pore pressure test operations include a second test operation subsequent to the first test operation, the second test operation including operating the first pump to circulate the test fluid from the first reservoir, through the flow inlet, and to the core sample in the core sample assembly at a third inlet pressure different than the first and second inlet pressures, operating the second pump to circulate the test fluid from the core sample in the core sample assembly at the second outlet pressure, through the flow outlet, and to the second reservoir, and operating the pressurized fluid pump to circulate the pressurized fluid from the pressurized fluid reservoir to the pressurized container at the second confining pressure.

In another aspect combinable with any of the previous aspects, the at least three high pore pressure test operations including a third test operation subsequent to the first and second test operations, the third test operation includes operating the first pump to circulate the test fluid from the first reservoir, through the flow inlet, and to the core sample in the core sample assembly at the second inlet pressure, operating the second pump to circulate the test fluid from the core sample in the core sample assembly at the second outlet pressure, through the flow outlet, and to the second reservoir, and operating the pressurized fluid pump to circulate the pressurized fluid from the pressurized fluid reservoir to the pressurized container at a third confining pressure different than the first and second confining pressures.

In another aspect combinable with any of the previous aspects, determining a permeability of the core sample based at least in part on at least one of the measured inlet pressures, at least one of the measured outlet pressures, and at least one of the measured confining pressures includes determining a pressure dependence coefficient of the core sample based at least in part on the second inlet pressure, the second outlet pressure, and the second and third confining pressures.

In another aspect combinable with any of the previous aspects, determining the pressure dependence coefficient of the core sample based at least in part on the second inlet pressure, the second outlet pressure, and the second and third confining pressures includes determining the pressure dependence coefficient based on: a first mass flow rate of the test fluid that is based on the second inlet pressure and the second outlet pressure, a second mass flow rate of the test fluid that is based on the second inlet pressure and the second outlet pressure, and a difference between the second and third confining pressures.

Another aspect combinable with any of the previous aspects includes determining a poroelastic coefficient of the core sample based at least in part on the pressure dependence coefficient.

What is claimed is:

1. A method for measuring rock permeability incorporating diffusion effects, the method comprising:
positioning a core sample in a core sample assembly that is enclosed in a pressurized container comprising a flow inlet, a flow outlet, and a pressurized fluid inlet fluidly coupled to a pressurized fluid reservoir that comprises a pressurized fluid pump;
performing a low pore pressure test operation on the core sample, the low pore pressure test operation comprising flowing a test fluid through the core sample between the flow inlet and the flow outlet with a first pore pressure, and maintaining a first confining pressure around the core sample by flowing a pressurized fluid into the pressurized container from the pressurized fluid reservoir;
sequentially performing at least three high pore pressure test operations on the core sample, each of the at least three high pore pressure test operations comprising flowing the test fluid through the core sample between the flow inlet and the flow outlet, and maintaining a second pressure around the core sample by flowing a pressurized fluid into the pressurized container from the pressurized fluid reservoir, wherein the second confining pressure is at least 2500 psi and the second pore pressure is at least 1000 psi more than the first inlet pressure;
for the low pore pressure test operation and each of the at least three high pore pressure test operations, measuring an inlet pressure at the flow inlet, measuring an outlet pressure at the flow outlet, and measuring a confining pressure within the pressurized container;
determining a pressure dependent coefficient α, a permeability with zero effective stress k₀, a poroelastic coefficient β, of the core sample based at least in part on the measured inlet pressures, the measured outlet pressures, and the measured confining pressures of the three high pore pressure test operations; and
determining a pore radius with zero effective stress r₀ of the core sample based on the determined α, k₀, and β, based at least in part on the measured inlet pressures, the measured outlet pressures, and the measured confining pressures of the low pore pressure test operation.

2. The method of claim 1, wherein the low pore pressure test operation comprises:
operating a first pump to circulate the test fluid from a first reservoir, through the flow inlet, and to the core sample in the core sample assembly at the first inlet pressure;
operating a second pump to circulate the test fluid from the core sample in the core sample assembly at a first outlet pressure, through the flow outlet, and to a second reservoir; and
operating the pressurized fluid pump to circulate a pressurized fluid from the pressurized fluid reservoir to the pressurized container at the first confining pressure.

3. The method of claim 2, wherein the at least three high pore pressure test operations comprises a first test operation that comprises:
operating the first pump to circulate the test fluid from the first reservoir, through the flow inlet, and to the core sample in the core sample assembly at the second inlet pressure,
operating a second pump to circulate the test fluid from the core sample in the core sample assembly at a second outlet pressure greater than the first outlet pressure, through the flow outlet, and to the second reservoir, and
operating the pressurized fluid pump to circulate the pressurized fluid from the pressurized fluid reservoir to the pressurized container at the second confining pressure.

4. The method of claim 3, wherein the at least three high pore pressure test operations comprises a second test operation subsequent to the first test operation, the second test operation comprising:
operating the first pump to circulate the test fluid from the first reservoir, through the flow inlet, and to the core sample in the core sample assembly at a third inlet pressure different than the first and second inlet pressures,
operating the second pump to circulate the test fluid from the core sample in the core sample assembly at the second outlet pressure, through the flow outlet, and to the second reservoir, and
operating the pressurized fluid pump to circulate the pressurized fluid from the pressurized fluid reservoir to the pressurized container at the second confining pressure.

5. The method of claim 4, wherein the at least three high pore pressure test operations comprises a third test operation subsequent to the first and second test operations, the third test operation comprising:
operating the first pump to circulate the test fluid from the first reservoir, through the flow inlet, and to the core sample in the core sample assembly at the second inlet pressure,
operating the second pump to circulate the test fluid from the core sample in the core sample assembly at the second outlet pressure, through the flow outlet, and to the second reservoir, and
operating the pressurized fluid pump to circulate the pressurized fluid from the pressurized fluid reservoir to the pressurized container at a third confining pressure different than the first and second confining pressures.

6. The method of claim 5, wherein the pressure dependence coefficient α of the core sample is determined based at least in part on the second inlet pressure, the second outlet pressure, and the second and third confining pressures.

7. The method of claim 6, wherein determining the pressure dependence coefficient α of the core sample based at least in part on the second inlet pressure, the second outlet pressure, and the second and third confining pressures comprises:
determining the pressure dependence coefficient α based on:
a first mass flow rate of the test fluid that is based on the second inlet pressure and the second outlet pressure,
a second mass flow rate of the test fluid that is based on the second inlet pressure and the second outlet pressure, and
a difference between the second and third confining pressures.

8. The method of claim 6, wherein the poroelastic coefficient β of the core sample is determined based at least in part on the pressure dependence coefficient α.

9. The method of claim 8, wherein determining the poroelastic coefficient β of the core sample based at least in part on the pressure dependence coefficient comprises:
determining a product of the poroelastic coefficient β and the pressure dependence coefficient α based at least in part on:
a first mass flow rate of the test fluid that is based on the second inlet pressure and the second outlet pressure,
a second mass flow rate of the test fluid that is based on the second inlet pressure and the second outlet pressure,
the second and third inlet pressures,
the second outlet pressure, and
the second confining pressure; and
determining the poroelastic coefficient β from the product of the poroelastic coefficient β and the pressure dependence coefficient α.

10. The method of claim 8, further comprising determining the permeability of the core sample based at least in part on the poroelastic coefficient β and the pressure dependence coefficient α.

11. The method of claim 9, wherein determining the permeability of the core sample based at least in part on the poroelastic coefficient β and the pressure dependence coefficient α comprises determining the permeability of the core sample based at least in part on:
a first mass flow rate of the test fluid that is based on the first inlet pressure and the first outlet pressure;
a cross-section area of the core sample;
the poroelastic coefficient β;
the pressure dependence coefficient α;
a diffusion correction factor;
a density of the test fluid;
a viscosity of the test fluid;
a distance between the flow inlet and the flow outlet;
the first confining pressure;
the first outlet pressure; and
the first inlet pressure.

12. The method of claim 1, wherein each of the measured inlet pressure, the measured outlet pressure, and the measured confining pressure is at a steady state condition.

13. A system for determining a rock property, comprising:
a core sample assembly enclosed in a pressurized container that includes a flow inlet, a flow outlet, and a pressurized fluid inlet, the core sample assembly configured to secure a core sample;
a pressurized fluid reservoir that comprises a pressurized fluid pump, the pressurized fluid reservoir fluidly coupled to the pressurized fluid inlet;
a first test fluid reservoir that comprises a first pump, the first test fluid reservoir fluidly coupled to the flow inlet;
a second test fluid reservoir that comprises a second pump, the second test fluid reservoir fluidly coupled to the flow outlet;
a plurality of fluid sensors, at least one fluid sensor positioned at or near each of the flow inlet, the flow outlet, and the pressurized fluid inlet; and
a control system communicably coupled to the plurality of fluid sensors, the pressurized fluid pump, the first pump, and the second pump, the control system configured to perform operations comprising:
operating the pressurized fluid pump, the first pump, and the second pump to perform a low pore pressure test operation on the core sample and to sequentially perform at least three high pore pressure test operations on the core sample, each of the test operations comprising flowing a test fluid into the flow inlet from the first test fluid reservoir and flowing the test fluid out of the flow outlet into the second test fluid reservoir, and flowing a pressurized fluid into the pressurized container from the pressurized fluid reservoir;
for the low pore pressure test operation and each of the at least three high pore pressure test operations, receiving, from the plurality of fluid sensors, measurements comprising an inlet pressure at the flow inlet, an outlet pressure at the flow outlet, and a confining pressure within the pressurized container; and
determining a permeability of the core sample based at least in part on at least one of the measured inlet pressures, at least one of the measured outlet pressures, and at least one of the measured confining pressures.

14. The system of claim 13, wherein the low pore pressure test operation comprises:
operating a first pump to circulate the test fluid from a first reservoir, through the flow inlet, and to the core sample in the core sample assembly at a first inlet pressure;
operating a second pump to circulate the test fluid from the core sample in the core sample assembly at a first outlet pressure, through the flow outlet, and to a second reservoir; and
operating the pressurized fluid pump to circulate a pressurized fluid from the pressurized fluid reservoir to the pressurized container at a first confining pressure.

15. The system of claim 14, wherein the at least three high pore pressure test operations comprises a first test operation that comprises:
operating the first pump to circulate the test fluid from the first reservoir, through the flow inlet, and to the core sample in the core sample assembly at a second inlet pressure greater than the first inlet pressure,
operating a second pump to circulate the test fluid from the core sample in the core sample assembly at a second outlet pressure greater than the first outlet pressure, through the flow outlet, and to the second reservoir, and
operating the pressurized fluid pump to circulate the pressurized fluid from the pressurized fluid reservoir to the pressurized container at a second confining pressure greater than the first confining pressure.

16. The system of claim 15, wherein the at least three high pore pressure test operations comprises a second test operation subsequent to the first test operation, the second test operation comprising:
operating the first pump to circulate the test fluid from the first reservoir, through the flow inlet, and to the core sample in the core sample assembly at a third inlet pressure different than the first and second inlet pressures,
operating the second pump to circulate the test fluid from the core sample in the core sample assembly at the second outlet pressure, through the flow outlet, and to the second reservoir, and
operating the pressurized fluid pump to circulate the pressurized fluid from the pressurized fluid reservoir to the pressurized container at the second confining pressure.

17. The system of claim 16, wherein the at least three high pore pressure test operations comprises a third test operation subsequent to the first and second test operations, the third test operation comprising:
operating the first pump to circulate the test fluid from the first reservoir, through the flow inlet, and to the core sample in the core sample assembly at the second inlet pressure,
operating the second pump to circulate the test fluid from the core sample in the core sample assembly at the second outlet pressure, through the flow outlet, and to the second reservoir, and
operating the pressurized fluid pump to circulate the pressurized fluid from the pressurized fluid reservoir to the pressurized container at a third confining pressure different than the first and second confining pressures.

18. The system of claim 17, wherein determining a permeability of the core sample based at least in part on at least one of the measured inlet pressures, at least one of the measured outlet pressures, and at least one of the measured confining pressures comprises:
determining a pressure dependence coefficient of the core sample based at least in part on the second inlet pressure, the second outlet pressure, and the second and third confining pressures.

19. The system of claim 18, wherein determining the pressure dependence coefficient of the core sample based at least in part on the second inlet pressure, the second outlet pressure, and the second and third confining pressures comprises:
determining the pressure dependence coefficient based on:
a first mass flow rate of the test fluid that is based on the second inlet pressure and the second outlet pressure,
a second mass flow rate of the test fluid that is based on the second inlet pressure and the second outlet pressure, and
a difference between the second and third confining pressures.

20. The system of claim 18, further comprising determining a poroelastic coefficient of the core sample based at least in part on the pressure dependence coefficient.
